(19) United States
(12) Patent Application Publication
(10) Pub. No.: US 2019/0056409 A1
(43) Pub. Date: Feb. 21, 2019

(54) Pooling by Allele Partition Designs For Identifying Anti-HLA and Other AlloAntibodies and Selecting Cell and Graft Donors

(71) Applicant: BioInventors & Entrepreneurs Network, LLC, Warren, NJ (US)

(72) Inventor: Michael Seul, Basking Ridge, NJ (US)

(21) Appl. No.: 16/105,290

(22) Filed: Aug. 20, 2018

Related U.S. Application Data
(60) Provisional application No. 62/547,780, filed on Aug. 19, 2017.

(51) Int. Cl. G01N 33/68; B01L 3/00

(52) U.S. Cl. CPC G01N 33/6854; B01L 3/50855

(57) ABSTRACT

The invention relates to forming locus-specific (or gene-specific) pools of cells from various potential donors, where the cells that express or display HLA molecules, or other polymorphic cell surface markers, and identifying the pattern of responses across these pools, notably aggregation of cells, or their acquisition of fluorescence when exposed to a labeled secondary antibody, when exposed to sera from a potential recipient. Pools of cells from different potential donors are designed and constructed in accordance with allele partitions that are identified by amino acid sequence tags, preferably defined in terms of single or combinations of variable amino acid positions encoded by the constituent alleles in the partition.

### BACKGROUND

Anti-HLA antibodies and other antibodies such as those directed to polymorphic antigens expressed on red cells including RHCE and RHD may be formed in response to allo-exposure, that is: exposure to foreign antigens or antigenic determinants, primarily as a result of pregnancy, transfusion of cells such as platelets (TRAP1997), red cells (Everett1986, Leffell2014), leukocytes or stem cells, as well as organ transplantation (Einecke2009).

While screening for allo-antibodies directed to some of the common antigens in the RH blood group is standard practice for pre-transfusion work-ups, antibodies to less common antigenic determinants largely go unchecked, in part because of a lack of suitable reagents, and the complexity and cost of current laboratory procedures. The complexity arises because the high degree of polymorphism of the genes encoding the RHCE and RHD antigens such that each of these molecules can display multiple antigenic determinants. This illustrates the larger problem of characterizing polymorphic antigens expressed on cell surfaces (be they circulating cells, such as red cells of platelets, or tissue cells such as epithelial cells), Human Leukocyte Antigens (“HLA”), encoded by the most polymorphic locus in the genome, therefore represent the ultimate challenge in this regard.

Anti-HLA antibodies are monitored routinely for organ and stem cell transplant candidates to exclude, in so far as practical, donors of cells or grafts that would expose the prospective recipient to foreign HLA antigens (“allo-exposure”), a factor that, for kidney graft recipients, has been shown to increase the risk of antibody-mediated rejection (Sellares2012). Hematology or sickle cell anemia patients, notwithstanding the high proportion of sensitized patients in these populations (TRAP997, Sintnicolass1995, McPherson2010, Nickel2015). are not routinely monitored for anti-HLA antibodies.

An indication of the anti-HLA antibody status of organ transplant candidates is obtained by exposing patient sera to a panel of lymphocytes and counting the number of “positive” wells in a tray comprising different cells and determining a “panel-reactive antibodies” (aka “PRA”) score. The sore represents the proportion of a prospective graft donor population to which a would-be recipient is expected to mount an adverse reaction via pre-existing allo-antibodies (see also USDHHS website, Organ Procurement and Transplantation Network page).

The identification of ant-HLA antibodies initially relied predominantly on cell-based, complement-mediated “lymphocytotoxicity” assays (Fidler2012). While these have the advantage of detecting antibodies by their actual effect on a relevant Immunological pathway, they thereby also limit detection to complement-binding (“fixing”) subclasses of antibodies (though arguably it is these that are of primary clinical concern). To overcome this limitation, and that of limited detection sensitivity, ELISA formats were subsequently introduced (e.g. Buelow1995, One Lambda LAT package insert).

The currently prevalent assay format is one that permits the simultaneous detection of anti-HLA antibodies directed to locus-specific sets of single recombinant (Pei2003) or native (U.S. Pat. Nos. 8,927,290; 9,354,238) antigens, displayed on color-encoded polymeric beads. These “single-bead” assays, though they have the advantage of simultaneously probing for a multiplicity of locus-specific antibodies, with what is said to be superior sensitivity, are also well recognized to display substantial lot-to-lot, site-to-site and operator-to-operator variability (Zachary2009, Reed2013), to wit:
(i) as with most bead-based assays, non-specific interactions between antigens (often in the form of membrane fragments or extracts) and the polymeric bead surface (note: commercially prevalent beads include carboxylated or aminated polystyrene polymeric beads) can lead to partial or complete denaturation of the protein: a well-known and significant problem that affects specificity as well as sensitivity:
(ii) exposure of these beads to patient sera, which often contains many unrelated products including proteins and metabolites, that often is characterized by significant non-specific interactions of beads and serum constituents that not only increases the background signal recorded from these beads but also may interfere with the specific antigen-antibody interaction of interest;
(iii) the fact that the prevalent commercial implementations of this assay design relies on a second antibody for visualization of the primary antibody binding, brings with it all the well-known additional difficulties (e.g. the “hook” effect); thresholds for the recorded Mean Fluorescence Intensity (“MFI”) are set in ad-hoc fashion, by “consensus”, with substantial variability across areas of application (heart, liver, kidney transplantation) as to what is, and is not, considered a significant MFI level.

Thus, even under controlled conditions (same lot, same samples, same protocols), lab-to-lab variability is substantial. Further, interpreting Mean Fluorescence Intensity (“MFI”), recorded over bead sets, as a proxy for “antibody strength,” ignores the fact that the affinity constants for different antibody-antigen interactions may vary substantially while reflecting the binding of one or more antibodies in the serum to one or more beads, rendering the relationship between fluorescence intensity and antibody concentration in the serum unreliable.

Finally, notwithstanding the widespread practice in referring to antibody specificities in terms of the allele(s) displayed on “single-antigen” beads that turn positive when exposed to a patient serum, even a single positive bead, indicating a single responding allele, generally leaves substantial ambiguity as to the identity of the antibody, as each HLA allele encodes a multiplicity of antigenic determinants. For other polymorphic antigens or cell surface markers of interest, the analog of “single-antigen beads” may not exist, as it may be impractical to reliably manufacture beads with intact membrane proteins (such as RHCE or RHD).

For HLA, few attempts have been made to identify these antigenic determinants, a problem that is addressed by a method disclosed in a co-pending provisional application to which priority is claimed (No. 62/547,778) that has been otherwise considered in only a small number of references of note including El-Awar2009 and Sasaki & Castro (US Publ'n No. 2008/0091357).

The Terasaki Research Foundation (El-Awar2009) has published compilations of antigenic determinants identified by a characterized patient sera or by monoclonal antibodies, using a nomenclature for these determinants that is substantially retained here (as elaborated below). Sasaki & Castro (US Publ′n No. 2008/0091357) have described a computational approach that is based on the construction of data tables from combinations of amino acids in variable positions.

While this bears a conceptual resemblance to the construction of an allele partition dictionary, as disclosed in a co-pending provisional application (No. 62/547,778) to which priority is claimed, the authors appear to identify the combinations of amino acids with actual epitopes, in the same vein as does the work in ElAwar2009, but in contrast to the methods disclosed herein. Duquesnoy has proposed a definition of “epitopes” in terms of successive triplets of amino acids that, over the past decade or more, has produced mixed results (see e.g. Nambiar2006). While it is conceivable that this approach may (at best) identify linear epitopes, it would, by construction, miss any others including many of those described in El-Awar2009.

None of the foregoing references disclose the construction of pools of cells in accordance with an allele partition design for identifying antigenic determinants recognized by antibodies in the sera of prospective recipients of cells or grafts, and for selecting cells or grafts on the basis of the antigenic determinants expressed, or not expressed, on donor cells or grafts.

In view of these shortcomings and conceptual difficulties, existing testing methodology informing important clinical decisions, such as the selection of cells or organ grafts, must be considered less than robust, and raise an urgent need for a method that identifies antigenic determinants of antibodies in the patient sera.

### SUMMARY

The invention relates to a device and method for identifying antigenic determinants on HLA or other polymorphic antigens that are recognized by antibodies present in patient sera, or fluid media derived therefrom. The invention encompasses forming locus-specific (or gene-specific) pools of cells (or vesicles or membrane fragments derived therefrom) that express or display HLA molecules, or other polymorphic cell surface markers (such as RHCE or RHD, expressed on red blood cells, or other expressed proteins encoded by polymorphic genes) and identifying the pattern of responses across these pools when exposed to sera, notably aggregation of cells, or their acquisition of fluorescence when exposed to a labeled secondary antibody. These response patterns to the serum of a specific prospective recipient of cells or an organ graft reveal the identity of any allo-antibody in the serum by identifying its cognate antigenic determinant, and that information is then used to identify suitable donors, and to exclude unsuitable donors for that recipient.

Pools of cells are designed and constructed in accordance with allele partitions that are generated by amino acid sequence tags, preferably defined in terms of combinations of j (j 1) variable positions in the amino acid sequence for the antigens of interest, as disclosed herein in detail for HLA loci. Each such tag serves as a unique identifier for a specific antigenic determinant (though it may not represent its actual physical manifestation, as argued, for example, in El-Awar2009). The pools produced by the method disclosed herein each represent a specific allele partition and each contains cells that all share one specific antigenic determinant expressed by all the alleles in the partition.

The preferred embodiment of the invention invokes antibody-mediated aggregation (aka “agglutination”) of cells to assess the presence of absence of a cognate antigenic determinant in pools. Antibody-mediated aggregation also is the basis of standard “cross-match” assays to establish the compatibility of donor cells with recipient serum (see laboratoryinfo.com website, at the “Cross-Matching: Types, Purpose, Principle, Procedure and Interpretation” page), and thus the preferred embodiment of the invention also permits the direct selection of “antigen-negative” cells and grafts (that is, cells or grafts not presenting a cognate antigenic determinant to existing antibodies) which, under that definition, would be considered suitable for administration to an intended recipient. In that sense, the method of the invention also represents a “pooled” (hence parallel) format of a serologic “cross-match” for rapidly identifying desirable candidate donors of cells or grafts.

An antibody will agglutinate (substantially) ALL the cells that express a cognate antigenic determinant, though a secondary antibody may be required to facilitate agglutination (an effect well known in the literature as the “Indirect Coombs Test”). Accordingly, an antibody is expected to agglutinate all the cells in a pool representing the allele partition generated by the aaTag identifying its cognate antigenic determinant (and any of its sub-partitions); while the same antibody will agglutinate only those cells in other pools that express the cognate antigenic determinant, while leaving other cells in suspension, and will not agglutinate cells in pools not containing any cells displaying the cognate antigenic determinant. Accordingly, the pool aggregation patterns produced by an antibody, or antibodies, in a serum will be characterized by a range of residual turbidities across a set of pools constructed in accordance with the allele partitioning design of the invention. Thus, a set of, say 16 or 48 or 96 or 384 such pools will yield multiple informative data points facilitating the identification of cognate antigenic determinants.

In another embodiment, a labeled secondary antibody, targeting antibodies in the serum, is used to aid in characterizing or quantifying the degree of aggregation. In such case, the corresponding fluorescence patterns to (i) immediately above (all cells agglutinated) is: ALL cells fluorescent; and the corresponding fluorescence pattern to (ii) above is that some cells are fluorescent; and, for case (iii) above, no cells are fluorescent. In such embodiment, the secondary antibodies are preferably directed to the Fc fragment of the antibodies bound to the cells (most commonly in the IgG class), and these secondary antibodies are labeled with a fluorescent dye in accordance with standard practice in immunodiagnostics.

The HLA type (that is: the HLA encoding alleles) of the recipient, or corresponding “type” relating to other antigens, can be taken into account in generating a “personalized” pool design, because all the antigenic determinants encoded by the recipient's own alleles (“self-antigens” or “self-antigenic determinants”) can be excluded from consideration, as allo-antibodies, by definition, will be formed only to “foreign” antigens or antigenic determinants. Thus, the invention identifies antibodies present in the sera by their interaction with the actual cognate antigenic determinants expressed on pooled cells.

Because the cells considered here generally will express a multiplicity of antigenic determinants at each HLA locus, the use of such cells for the identification of antigenic determinants would appear to be impractical given the inherent ambiguities. While the failure of a serum to agglutinate a first type of cell of known HLA profile permits the exclusion of the antigenic determinants expressed on that cell as the target for any antibodies that might be present in the serum, a positive result, beyond indicating the presence of at least some allo-antibody, would leave the identity of any such antibody indeterminate, as that antibody could have bound to any cognate antigenic determinant on any of the HLA molecules expressed at any locus.

The construction of pools in accordance with the methods of the invention overcomes this problem (with certain exceptions, as elaborated herein). The pool designs disclosed herein are informed by a dictionary of allele partitions generated by variable amino acid positions in the mature protein sequence at each gene locus, as well combinations of two or more such variable positions. These combinations of amino acids at one or more designated variable amino acid positions serve as unique “tags” for the partitions they generate and thereby as a unique identifier of the corresponding antigenic determinant. An algorithm for constructing an allele partition dictionary also is disclosed herein.

In contrast to existing methods for anti-HLA antibody detection and identification, the method of the invention identifies the actual antigenic determinant recognized by antibodies (rather than merely giving specificities in terms of allele names). The method comprises preparing pools of cells in accordance with allele partitions, and placing sera (or fluids derived therefrom) from an individual that is suspected to contain anti-HLA antibodies (or other antibodies directed to antigens with multiple antigenic determinants such as RHCE or RHD for red cells), in contact with the constituent cells in each pool, and determining the degree of antibody binding to these cells. Some embodiments also may use, in lieu of cells, affinity-purified or recombinant HLA such as those used in commercial single-bead assays, which may be displayed on microparticles (for example core-shell microparticles of the type disclosed in U.S. Pat. No. 6,964,747 “Preparation of Dyed Polymer Microparticles”) prepared as to minimize the risk of denaturing such displayed proteins. In one such embodiment, locus-specific HLA are reconstituted into phospholipid vesicles (Engelhard1978).

In a preferred embodiment, cells or vesicles or membrane fragments derived therefrom are in stable suspension permitting the detection of antibody-mediated aggregation. In some embodiments, aggregation is mediated by a secondary antibody (as in the Indirect Coombs Test). Aggregation above a preset threshold in any pool indicates the presence of the antibody in the serum. In a preferred embodiment, the degree of aggregation of a pool is determined by way of a quantitative determination of the clearing of the suspension. This may be accomplished by many methods, for example by optical interrogation, preferably in a plate reader (by measuring turbidity or, equivalently, optical density) or in a flow cytometer (by measuring side scatter). Additional methods for assessing the degree of agglutination may also be used, as well as commercial methods, including the “Capture” assay (Immucor) or gel-filtration assay (Diamed).

The degree of aggregation is used to identify cognate antigenic determinants recognized by an antibody, or antibodies, in a serum, and that information is used in turn to either identify suitable donor for the individual contributing the serum (aka a prospective “recipient”), or to form a database of cognate antigenic determinants which can be referenced, and used to form pools of cells, for other recipients. The degree of aggregation preferably is used to rank the pools, from highest to lowest degree of aggregation. The ranking can be compared to a predicted ranking (based on the cells' allele composition) in order to confirm or further refine the identification the cognate antigenic determinants. This information also may inform the refinement of pool designs.

In a preferred embodiment, testing on each pool is performed in triplicate, and then the observed degree of aggregation is averaged.

In another embodiment, cells expressing only class I but not class II HLA also can be used to fractionate sera by class, namely by removing anti-class I antibodies, or conversely, to ignore anti-class II antibodies by constructing pools excluding class II expressing cells. Similarly, cells from RHD-negative donors can be used to fractionate sera by removing antibodies directed to RHCE.

The present invention for identifying allo-antibodies by their cognate antigenic determinant provides guidance for therapeutic intervention such that, in the presence of any allo-antibody, further allo-exposure to cognate antigenic determinants, be they displayed on cells given to anemic or thrombocytopenic patients or on organ or stem cell grafts given to transplant candidates, can be avoided, so as to reduce the adverse effect of antibody-mediated clearance of cells or rejection of grafts. The present invention directly identifies cells (and by extension grafts displaying suitable “antigen-negative” epithelial cells) suitable for administration to intended recipients as those in non-aggregated or minimally aggregated partitions.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1: Schematic illustration of allele partitions—HLA molecules, labeled by C-allele names, are indicated as lines (emanating from cells), each expressing one or more of the 7 antigenic determinants indicated by different geometric shapes (“pearls on a string”): the vertical boxes indicate patterns of shared antigenic determinants over the allele set.

FIG. 2: A set of 16 common C-alleles, each specified by a letter identifying the locus and 4 digits identifying the allele.

FIG. 3: A first set of C-locus allele partitions for a set of common C-alleles (shown in FIG. 2), generated by single variable positions in the mature amino acid sequence encoded by each allele: rows are labeled by allele names, and columns by variable amino acid positions; the shaded row names indicate alleles with at least one “private” antigenic determinant; shown below each column are allele counts for the partitions defined by each of the variable amino acid “tags”; the total number of partitions generated; and the corresponding entropy by which columns are ordered. The generalization of this construction, to combinations of variable amino acid positions, is elaborated in the Detailed Description.

FIG. 4: With the same rows and column labels as in FIG. 3, this table shows an example of allele partitions generated by j=2 antigenic determinants comprising 77S and 77N (third shaded column).

FIG. 5: Exclusion of “self”-antigenic determinants encoded by allele C0202 and C0602: the non-shaded fields represent the allo-antigenic determinant profile (see also FIG. 3).

FIG. 6: Example of expected pool aggregation patterns for anti-“C:77S” and anti-“C:77N.”

FIG. 7: Example of expected pool aggregation patterns for anti-“C:97M” and anti-“C:97W.”

FIG. 8: Illustration of “By-Stander Aggregation” in A locus partitions by complete aggregation of C locus partition C:156R.

FIG. 9: Illustration of process flow for analyzing sera by HLA class.

### DETAILED DESCRIPTION

### Definitions

aaSeqTag (aka “j-aa tag” or simply “aaTag”)—amino acid sequence tag having a combination of j amino acids at variable positions in the amino acid sequence, where typically 1≤j≤3; each aaSeqTag represents an allele partition;
allele partition—a subset of a set of alleles such that alleles in a partition either contain, or do not contain, a specific combination of one or more amino acids designated by an “aaSeqTag”; such a tag is said to generate the partition;
allele partition dictionary—a compilation of all allele partitions, permitting a look-up of allele partitions by their respective generating aaTags;
blood products—includes whole blood and cellular products derived from blood, including red blood cells, platelets, leukocytes and any other derived cellular products;
n-plet—a combination of amino acids at n variable positions in the amino acid sequence; for example, “77S” and “95L” are 1-plets, “77S-95L” and “77S-951” are 2-plets, etc; see also below;
antigenic determinant—the molecular moiety representing the binding site(s) recognized by an antibody;
cognate—as in “cognate antigenic determinant”; designates the antigenic determinant of a specific antibody; a cognate allele is one that encodes a cognate antigenic determinant; a cognate allele partition is a partition whose constituent alleles all encode a cognate antigenic determinant of interest;
serum—includes serum itself and proteins and antibodies derived from serum.

Cell Sources—

Cells such as platelets (which express HLA class 1 comprising the principal loci A, B and C; but not HLA class II comprising the principal loci DR, DQ and DP) or lymphocytes and stem cells (which express both HLA class I and class II) or epithelial cell precursors (e.g. Vermehren2002) may be obtained from peripheral blood. Other cells of interest, notably epithelial cells (Canet2012) may be isolated from tissue and grown in culture; or cells may be derived from precursor cells (including stem cells).

Allele Partitions—

The Major Histocompatibility Complex (“MHC”) which encodes Human Leukocyte Antigens (“HLA”), given its high degree of polymorphism, has a large number of HLA alleles (see ebi.ac.uk wesite, at the IPD-IMGT/HLA page). Each of these allele encodes a multiplicity of antigenic determinants, with each such antigenic determinant being the potential target of an alloantibody. However, the common representation of this diversity of antigenic determinants in terms of alleles (historically designated in terms of serologic groups, and later elaborated by molecular analysis) “mixes” the antigenic determinants so that, when an allo-antibody binds to an HLA molecule encoded by a specific allele, as in the commercial “single bead” assays, a positive signal associated with a specific allele generally will not identify the actual antigenic determinant targeted by antibody.

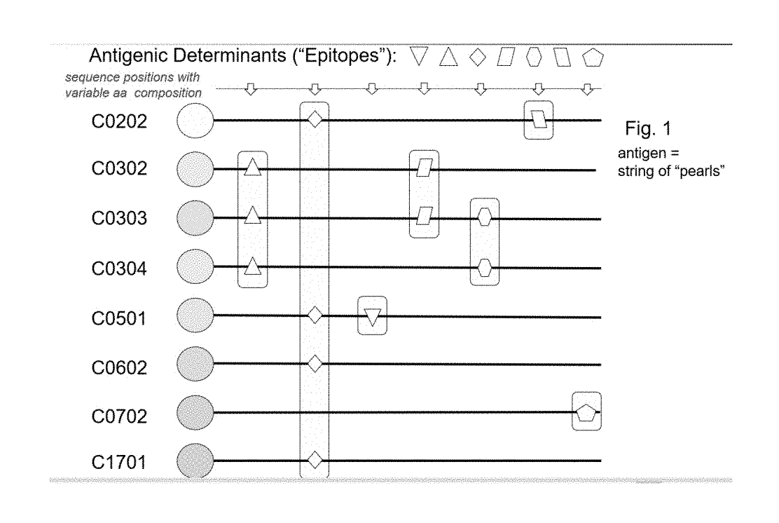

This is illustrated in FIG. 1, where a single allo-antibody directed to the antigenic determinant represented by the diamond shape (third from left at top of FIG. 1) would produce a positive signal for 4 beads, namely those labeled by C0202, C0501, C0602 and C1701, and leave the others negative. Conversely, a pattern having only 2 positive beads, namely C0202 and C0702, leaving all others negative, requires the presence of 2 allo-antibodies, as C0202 and C0702 have no common antigenic determinant. Thus, the binding patterns produced by single antigen beads, in order to support the correct determination of antibody status, require a decomposition in terms of true antigenic determinants.

The correct representation of the diversity of antigenic determinants is one that systematically groups alleles in terms of the antigenic determinants they share. On the assumption that all possible antigenic determinants are uniquely associated with combinations of j variable amino acid positions in the mature protein sequence, where typically 1 j 3, such a systematic grouping is constructed herein in the form of the set of allele partitions generated by these combinations of j variable amino acid positions. Alleles in a given partition share a specific antigenic determinant that is uniquely identified by the combination of j amino acids that generated that partition; these combinations are referred to herein as “j-aa tags.” Some of the antigenic determinants so identified coincide with those identified independently (El-Awar2009, Maiers2002).

Constructing Allele Partitions—

To construct partitions, proceed as follows:

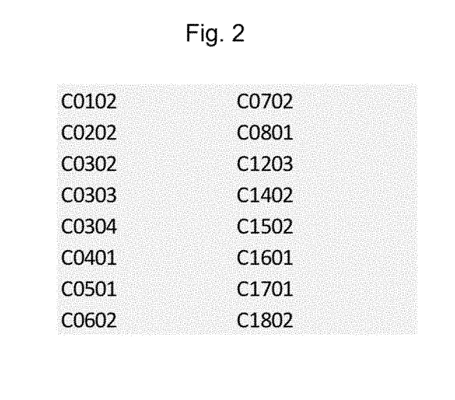

First, select alleles of interest at a specific locus (for example in accordance with the abundance of these alleles in a population of interest (such as the common C alleles listed in FIG. 2) and retrieve the corresponding amino acid sequences for the mature protein from a public database (notably the ebi.ac.uk website, at the IPD-IMGT/HLA page) and identify variable amino acid positions.

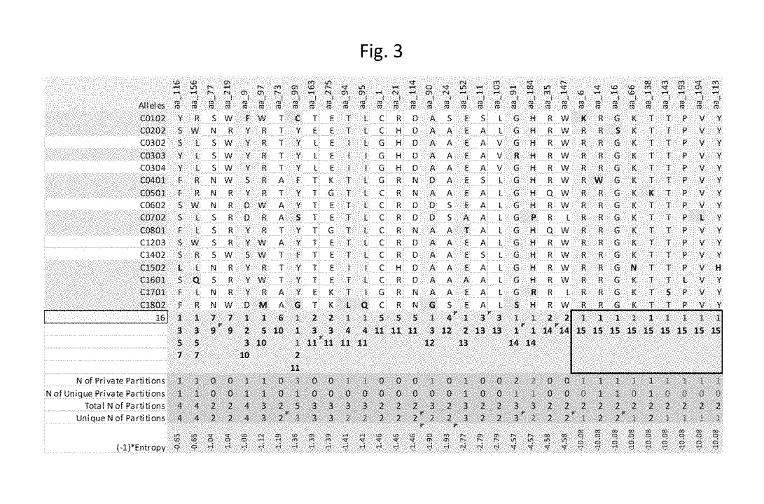

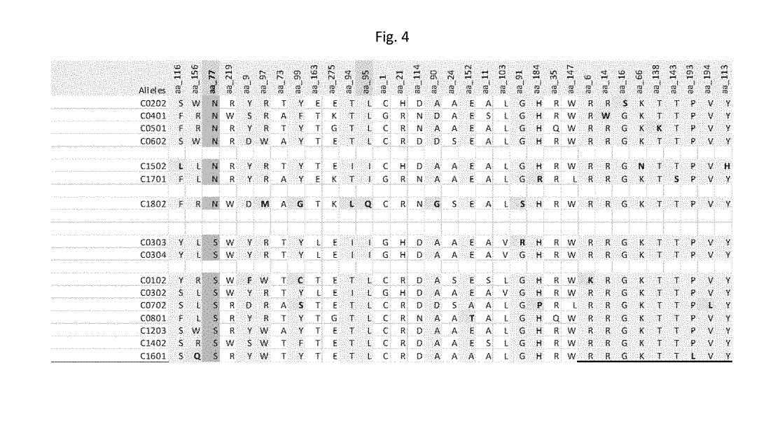

Next, construct all partitions induced by single (j=1) variable positions (as illustrated in FIG. 3) and by combinations of j=2 (as illustrated in FIG. 4) or combination of j=m variable amino acid positions (where typically m 3), in accordance with Algorithm I below.

A single variable amino acid position with k distinct amino acids (over the set of alleles included in the construction), defines k partitions of the allele set, wherein each partition contains at least 1 allele. The allele partitions defined at level j=1 by variable amino acid positions in the mature protein sequence for a set of 16 common C-alleles in FIG. 2 is illustrated in FIG. 3, where the 1-aaSeq tags labeling each column generate between k=2 and k=5 partitions. For example, the variable position 77, with amino acids N and S, generates the two partitions “77S” (comprising alleles with 77S), and “77N” (comprising alleles with 77N), while position 116 induces the 4 partitions “116L”, “116Y”, “116F” and “116S”.

Redundant partitions are generated by variable positions that are in complete linkage disequilibrium, and hence fully correlated: for example, for variable positions 77 and 80 (the latter not shown), 77S implies 80N (and vice versa) and 77N implies 80K (and vice versa). Redundant partitions, as well as the “trivial” partition comprising all alleles in the set, are excluded.

As HLA alleles typically differ from one another in multiple positions, each allele generally will be an element of multiple partitions. Conversely, the same antigenic determinant may be encoded by multiple alleles, as inspection of any row in the matrix of FIG. 3 will reveal.

Certain antigenic determinants are “private,” that is: encoded by only a single allele in the set, and the partition counts given below each column in FIG. 3 indicate 24 single allele partitions—the corresponding alleles are indicated by shaded row names (and shading of the relevant columns). Closer inspection reveals that several of the 1-aa sequence tags in fact encode the same single allele partition (e.g. 6 equivalent 1-aa tags labeling the single-allele partition C1802). Taking into account these degeneracies, the number of unique single allele partitions is found to be 11 and the total number of unique partitions, at level j=1, to be 69. Whether or not an antigenic determinant remains “private” when the underlying set of alleles is enlarged will of course depend on the structure of the HLA alleles in the population of interest.

The set of partitions so generated by individual variable amino acid positions (j=1) may be recursively extended by considering pairs (j=2) or n-plets (j=n) of variable amino acid positions, for example, for j=2, 77S-95L vs 77S-951, as illustrated in FIG. 4.

The complete set of partitions up to a preset j=m may be generated in accordance with the Algorithm I below, where the pseudocode is in the style of the R language (see also “R: A language and environment for statistical computing”). Only the steps up to j=2 are shown, extensions beyond j=2 would proceed accordingly (and recursive implementations are also possible).

Algorithm I

```
  genAllelePartitions <- function(aaProf,selVarAAPos)
{
    # return list of allele partitions, each partition comprising a subset of aSet in accordance
with shared aa for the selected element of vaaSet
    saP ← lapply(unique(aaProf[,selVarAAPos]), function(uaa)
rownames(aaProf)[which(aaProf[,selVarAAPos] %in% uaa),])
}
mgenAllelePartitions <- function(aaProf)
{
    # generate allele partitions of aaSet (=rownames (aaProf )) over varAASet
(=colnames(aaProf)
    aP ← lapply(vaaSet,function(selVarAAPos) genAllelePartitions(aaProf[,selVarAAPos])) # list
of lists
    aP <- flattenList(aP) # convert list (over vaaSet) of lists (over unique aa at selVarAAPos) into
simple list
    aP <- filter(isUnique, aP) # identify unique partitions (and record equivalent amino-acid
tags)
}
# SCRIPT
vaaSet = vector of strings representing set of variable amino acid positions in mature protein
seq at given locus
aSet = vector of strings representing set of selected alleles at a given locus (see e.g. FIG. 2),
aaProf = matrix of amino acid (“aa”) profiles for aSet over vaaSet (row names = aSet, column
names = vaaSet)
# construct allele partitions at j = 1: each allele partition, in the form of a subset of aaSet,
defined by single var aa positions
    aP_1 ← mgenAllelePartitions(aaProf) # list of partitions, each in the form of a vector of
allele names in aaSet
# construct allele partitions at j = 2: each allele partition defined by a pair of var aa positions
    aP_2 ← sapply(aP_1,function(saP) mgenAllelePartitions(aaProf[saP,]) ) # extended list of
partitions
```

The number of partitions increases rapidly with j: for example, for the 16 common C-alleles in FIG. 2, the respective counts for unique partitions are as follows:
1-aa: 69 (11); 2-aa: 458 (7); 3-aa: 1,409 (1),

where the numbers in parentheses represent allele partitions comprising only a single allele, indicating a “private” antigenic determinant (expressed by only that allele, as illustrated in FIG. 3).

By construction, the alleles in any partition generated by a specific j-plet share that specific combination of j amino acids in variable positions of the amino acid sequence. Consequently, each such j-plet (aka “aaSeqTag”) represents an identifying “tag” for the corresponding allele partition and thus a tag for the corresponding antigenic determinant. That is: each j-plet identifies an antigenic determinant, and thus the antibody directed to that antigenic determinant. Whether or not such a j-plet represents the actual molecular binding site has no bearing on the selection of use of the j-plet tag, or the design of the pooling.

Thus, these tags serve to index allele partitions, in the form of an allele partition dictionary holding these allele partitions; some partitions will be indexed by more than one j-aaSeqTag.

The method of the invention provides for the systematic extension of the allele partition dictionary, and a corresponding refinement in the desired resolution (that is: minimal permissible residual ambiguity) in identifying antigenic determinants, namely by increasing the number of constituent variable amino-acid positions that are combined into j-aaSeqTags for generating partitions, and by increasing the number of pools actually constructed for a specific test.

Allele partitions may also be constructed by this method for any other polymorphic protein of interest.

Pooling Designs—Allele partitions provide the basis for pooling designs permitting the direct experimental determination of antigenic determinants. Specifically, pools are constructed in accordance with a set of partitions such that at least one of the alleles of the (type of) cells in any partition must be one of the alleles in that partition. If the cognate partition is included in the design, then exposure to the corresponding antibody, if present, will, by construction, aggregate ALL cells in that partition (and those in any of its sub-partitions included in the design), but not in other partitions. This will be so regardless of whether or not the specific amino acids in a j-plet labeling the partition in fact represent actual binding sites (see also: El-Awar2009).

To limit the number of allele partitions and corresponding pools included in any design, a desired subset of alleles for constructing pools may be selected in accordance with the probability of allo-exposure to any antigenic determinant. For a determinant with population frequency f, say, the probability of at least 1 exposure in n transfusion events (where R is recipient and D is donor) is:

prob(f;n)=prob(R lacks agDet)*prob(at least 1D has agDet)=(1−f)*(1−(1−f)n)

This function, for given n, has a maximum at f*=exp(log(1/(n+1))/n), hence f*(n=1)=0.5, f*(n=2)=0.423,I*(n=4)=0.331,I*(n=8)=0.240,f*(n=16)=0.162; that is, with increasing number of transfusions, allo-antigenic determinants of increasingly LOWER abundance in the population represent the highest allo-exposure risk; however, as the probability of allo-exposure must of course vanish for f=0, the function, for large n, drops off very steeply for f<f*. Accordingly, for antigenic determinants of equal immunogenicity (that is, potency of inducing antibody formation upon allo-exposure), the expected abundance of allo-antibodies will increase with decreasing frequency of the cognate antigenic determinant, but then decrease rapidly.

As a general design consideration, partitions representing less frequent antigenic determinants will be given preference in pool designs for probing sera from multi-transfused individuals, while, for post-transplant monitoring of antibodies to implanted grafts, such preference would be given to antigens of intermediate abundance. Within a given patient population, allo-immunization risk represented by antigenic determinants encoded by very common (f>>f*(n)) or very rare (f<f*(n)) alleles will be low. These general considerations may be augmented by such empirical information as may be known, or become known, regarding the actual immunogenicities of specific antigenic determinants: for example, some antigenic determinants of weak immunogenicity will be expected to induce antibody formation with lower probability than otherwise expected. In this manner, pool designs are preferably iteratively refined.

Estimates of the expected sample size for constructing partitions, that is: the expected number of randomly collected red cell units, say, required for manufacturing a pool set—may be based on the least common allele in the selected allele set; this is further illustrated in the Examples. Access to several hundred different sample units for manufacturing should suffice for most practical purposes.

In one embodiment, for the case of HLA antibodies, cells in the consanguineous reference panel offered by IHWG Cell and DNA Bank inventory on the FredHutch.org website may be used: these cell lines are homozygous at A, B, C and DR loci.

Antibody Detection (“Screening”)—

Aggregation in any partition, even without interpretation of the aggregation or fluorescence patterns, is an indication of allo-antibodies directed to cognate antigenic determinants expressed on cell surfaces including HPA or HLA class I on platelets, red cell antigens or HLA class I on red cells, HLA class I or class II on leukocytes or epithelial cells (or other cognate antigenic determinants which are expressed on cell surfaces).

Antibody Identification—

In order to identify an antibody in a serum (or other fluid) by its cognate antigenic determinant, proceed, for each gene or gene locus, as follows:
pool cells in accordance with the locus-specific allele partition dictionary constructed in accordance with the process in Algorithm I under conditions producing a stable suspension at a concentration producing a desired (preset) level of turbidity, wherein each cell type may contribute two alleles (provided that at least one is in the selected allele partition);
expose suspended cells (or membrane fragments or vesicles derived therefrom) to the serum suspected to contain one or more antibodies, under conditions facilitating antibody-mediated aggregation of the cells; and, as necessary or desirable, expedite the aggregation process, e.g. by centrifugation; OPTIONALLY (for example in cases involving antigenic determinants present on the cell surface at low density) add a secondary antibody to mediate aggregation (see also Indirect Antiglobulin Test—as described on the Wikipedia website on the “Coombs Test” page);
monitor the turbidity of the suspension as aggregation proceeds, or determine the decrease in turbidity after a preset elapsed time; preferably, this may be done by standard methods implemented in commercial spectrophotometers that are set up to handle 96-well or 384-well or other plate formats; optionally, aggregation also may be monitored or assessed visually (as in several commercial methods for serologic blood group determination—see e.g. Immucor, Norcross, Ga.; Diamed, Canton, Ohio and others); and
rank pools by residual turbidity (or equivalently by decrease in turbidity or equivalent measure of the degree of aggregation): this ranking may be compared with predictions reflecting the allelic composition and thus the fraction of cells in each pool expressing a specific antigenic determinant of interest.

Only the cells in the allele partition labeled by the j-aa sequence tag corresponding to the cognate antigenic determinant(s) will undergo (substantially) complete aggregation. Cells in allele partitions comprising one or more of the alleles in this cognate partition will undergo partial aggregation, while cells without any cognate alleles (that is, alleles in the cognate partition) will remain suspended. Thus, residual turbidities will range from unchanged (that is: same as before addition of the serum) to substantially clear.

Because the cells in any of the pools generally also will express HLA molecules encoded at other HLA loci, a serum containing an antibody directed to, say, an A-locus antigenic determinant, but no antibody directed to a C-locus antigenic determinant, nevertheless may produce partial aggregation in one or more of the C-locus pools. However, the identity of the C-locus (and B-locus) pools exhibiting partial aggregation mediated by an anti-A antibody will reflect the A-locus aggregation pattern, rather than the pool design at the C-locus. Further, in the absence of complete linkage disequilibrium, complete aggregation by an antibody directed to an antigenic determinant at another locus will be an event with a vanishingly small probability (see EXAMPLE).

The actual degree of residual turbidity will reflect the pool composition, as well as the Poisson distribution of counts of constituent cell types: as desired, multiple instances of each pool may be included in the assay to permit averaging.

The method of the invention has several critical advantages over the methods of the prior art, including these:
it identifies antibodies by their cognate antigenic determinant, rather than by allele-specificity, as in the prevalent “single bead” assays (OneLambda-Thermo Fisher Scientific, Waltham, Mass.; Lifecodes-Immucor, Norcross, Ga.), and allele specificity, except for the special case of private antigenic determinants (that is, those encoded by only one of the alleles in the set) generally leaves the true identity of the antibody/-ies indeterminate;
it avoids the introduction of polymeric (“bead”) surfaces that promote antibody denaturation and lead to spurious effects and undetermined levels of non-specific interactions between sera and bead surfaces that contribute to the high lot-to-lot, site-to-site and operator-to-operator variability of these assays that leaves thresholding of intensities (aka “MFI” values recorded from these beads in flow cytometric assays) open to significant uncertainty; and
in its preferred embodiment, it eliminates synthetic reagents from the assay, though these may be added to expedite or visualize aggregation by any of a number of available methods, well known in the art.

Pool Fluorescence Pattern—

In another embodiment, a fluorescently labeled secondary anti-IgG antibody may be used, in accordance with standard immunoassay formats, to visualize the presence of bound allo-antibodies on cell surfaces, and cell-associated fluorescence is determined by standard flow cytometry. Partial aggregation here will manifest itself in the shape of the distribution of fluorescence intensity over cells in each pool. Cells bearing allo-antibody, that is, those undergoing aggregation, will be fluorescent, while those not bearing allo-antibody (noaggregation) will not be fluorescent. Accordingly, pools undergoing complete aggregation will comprise only fluorescent cells, while pools undergoing partial aggregation will comprise both fluorescent and non-fluorescent cells, and pools undergoing no aggregation will produce no fluorescence (other than “background” fluorescence that may result from any non-specific adhesion of the labeled secondary antibody to cell surfaces).

AlloAntigen Profile as a Constraint in Pattern Matching—

As allo-antibodies will be formed to “non-self” (“allo-”) but not to “self” antigenic determinants, an intended recipient's allele profile for HLA preferably determined at the 4-digit level, as assumed in the Examples herein, provides an important constraint on the construction of pools as well as the interpretation of observed aggregation patterns. Specifically, by application of this principle, ALL antigenic determinants encoded by the intended recipient's alleles may be eliminated from consideration. For example, with reference to FIG. 3, we can exclude from consideration an anti-C:97M antibody for any intended recipient with allele C1802.

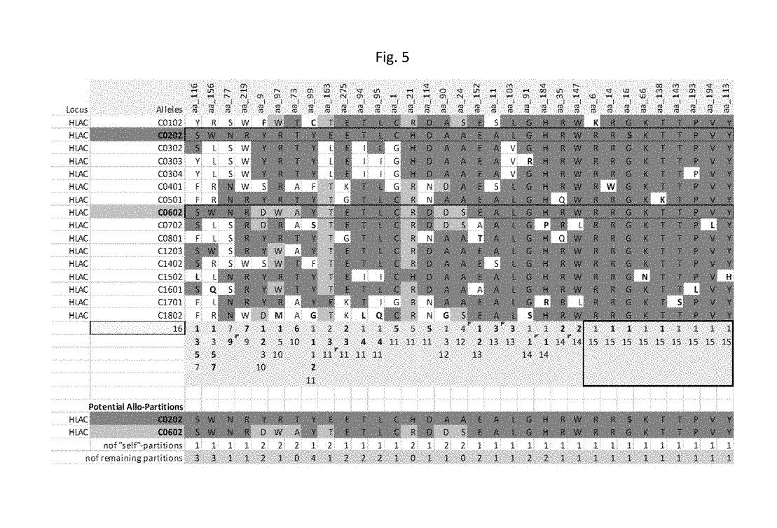

More generally, all antigenic determinants encoded by the intended recipient's alleles at any gene or gene locus, and in fact all allele partitions generated by any of these “self” antigenic determinants, can be eliminated from consideration. That this is a potent constraint is illustrated in FIG. 5 for a recipient with HLA alleles C0202-00602: the comparison of FIG. 5 to FIG. 3 shows that only 44 of the original 69 independent allele partitions remain in this case, as indicated by the unshaded fields in the matrix of Fig. S.

Producing Recipient-Specific (“personalized”) Pools—

This consideration motivates a further embodiment of the invention that will be particularly useful in a specialty laboratory setting, and that is to produce pool sets that only include allele partitions generated by “allo-antigenic determinants” of an intended recipient. These partitions are readily identified by lookup in an allele partition dictionary which lists allele partitions by unique j-aa tag and, for each, enumerates the set of constituent alleles so that a look-up of the antigenic determinants encoded by each such alleles identifies all “self” determinants, leaving only “allo”-determinants.

In a preferred embodiment, collections of cells for pool production, preferably in a plate with known plate map, and access to a software program for pool assembly would be provided, preferably in the form of as a web-hosted application. Given the HLA type of the intended recipient, the program will determine the allo-antigenic determinant profile of the intended recipient and will assemble pools accordingly. In a preferred embodiment, this would be accomplished by computing the pool design, and translating this into a downloadable script for a liquid handler that will generate pools on the fly—liquid handlers capable of downloading scripts from web-based script repositories are commercially available (see e.g. the opentrons.com website).

In another preferred embodiment of producing personalized pools, cells are added sequentially to the serum in a pool, where both alleles of any such cell belong to a known allele partition. Starting with a randomly chosen cell type, the aggregation response, which is now “binary” (aggregation; or not), is assessed after each addition. If the first cell type is aggregated, then this pool is expanded by further additions of different cell types, until encountering non-aggregation (which can be observed because prior aggregates settle out to the bottom of the pool). If the first cell type fails to be aggregated, this pool is expanded by further additions, until encountering aggregation. New pools may be started, as necessary, until aggregating and non-aggregating allele partitions have been defined to the desired degree.

Aggregation (or Fluorescence) Pattern Matching Algorithm—

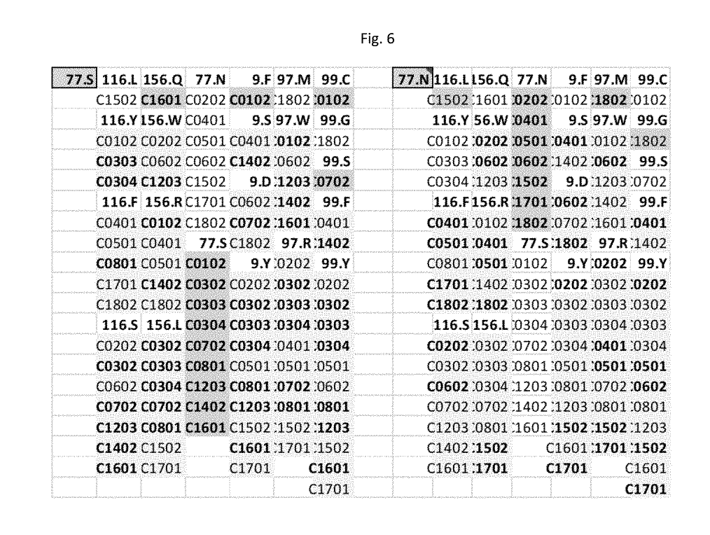

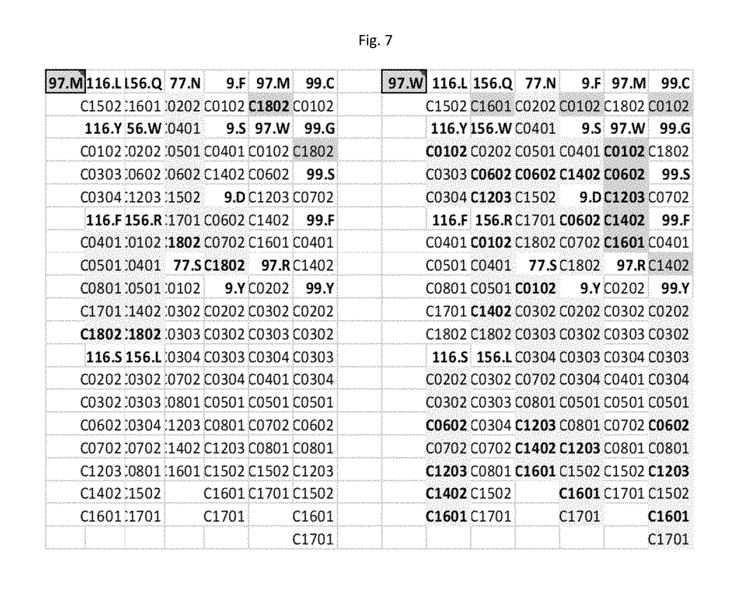

Given an allele partition dictionary, constructed by Algorithm I, and given an allele profile such as the HLA type of an intended recipient, determine the allo-partitions for that individual by a look-up in the allele partition dictionary; then, determine the complement of the set of j-aa tags for antigenic determinants encoded by the intended recipient's alleles at the locus of interest: this represents the intended recipient's allo-profile—that is, the recipient can form allo-antibodies only against the antigenic determinants in the set of allo-partitions. Next, for each of these allo-partitions, predict the aggregation (or fluorescence) pattern: the cognate partition (and any of its sub-partitions) will undergo complete aggregation (or display fluorescence for all constituent cells) whereas the complement(s) of the cognate partition (where the jaa tag for such cognate partition has a different amino acid at one or more of its tag sites) will undergo no aggregation (and display no fluorescence for any of the constituent cells). The remaining partitions will undergo partial aggregation (or display fluorescence on some, but not all constituent cells), where the degree of the aggregation (or fluorescence) reflects the fraction of alleles in the partition that express the cognate antigenic determinant; and, the larger that fraction, the higher the proportion of cells undergoing aggregation (or displaying fluorescence). This is the complete set of predicted aggregation (or fluorescence) patterns for single assumed allo-antibodies (aka “simple” or “pure”) patterns. This is illustrated in Examples which reference FIGS. 6 and 7.

Next, match the observed aggregation (or fluorescence) pattern against the set of the predicted patterns and choose the best match. In the absence of complete aggregation (or fluorescence: that is, all cells labeled), the set of partitions may be expanded to resolve the ambiguity. To account for observed patterns that do not yield an acceptable match to predicted patterns for single antigenic determinants (corresponding to a single allo-antibody), superpositions of m=2 or more patterns may be considered in a manner that seeks to minimize m.

Addressing Remaining Ambiguities:

Do No Harm—Inspection of the expected patterns, in FIGS. 6 and 7, immediately reveals that these pool designs would not permit the detection of an anti-97M antibody in the presence of an anti-77N antibody. In some instances, such “masking”—here of anti-97M by anti-77N—may be ruled, if an individual expresses the antigenic determinant labeled by “77N” and thus will not make an allo-antibody to that determinant (see e.g. FIG. 5).

In the general instance, “masking” may remain, as it does for commercial “single bead” assays which cannot detect an antibody directed to a first antigenic determinant, X, that is encoded by alleles that also encode a second antigenic determinant, Y: both anti-X and anti-Y antibodies will bind to the same set of single antigen beads and thus generate the same fluorescence pattern).

In general, the minimal therapeutic intervention would be to eliminate future allo-exposure to 77N by selecting a cell or graft that does not express this antigenic determinant, leaving the possible risk of allo-exposure to 97M.

Additional Strategies for Therapeutic Intervention—

Thus, the prudent therapeutic intervention will be to eliminate not only the allele partition 77N, but all its sub-partitions at j=2 which, as illustrated in FIG. 4, would eliminate the possibility of allo-exposure to all antigenic determinants encoded by C1802, including 97M. Even if anti-97M had not in fact been formed, this intervention would have the advantage of minimizing the risk of future sensitization to 97M. Thus, the optimal strategy would be to select cells in any non-aggregated pools

In the absence of non-aggregated pools, cells from pools with the lowest degree of aggregation (or smallest fraction of fluorescence) will be potential candidates: these may be found by breaking pools and exposing individual constituent cells to the intended recipient's serum

“By-Stander” Aggregation—

An allo-antibody directed to an antigenic determinant encoded at a first locus may induce aggregation in pools at a second locus, namely whenever the constituent cells in such pools, in addition to the alleles for which they were selected, also have alleles encoding a cognate antigenic determinant at the first locus. For example, an antibody directed to the C-locus determinant 77N will be expected to induce aggregation in all pools comprising cells with any of the C-alleles in the cognate partition; in this instance, for example, any A-locus pool including cells with at least one of these alleles: C0202, C0401, C0501, C0601, C1502, C1701, C1802 (see FIGS. 3 and 6). However, the aggregation patterns will be random with respect to the locus-specific pool design. Thus, one expects aggregation in any A-locus pool comprising the allele A0101 on cells that also express HLA encoded by alleles C0401 or C0601, say, but not in any pool that, while comprising A0101, does not comprise any of the cognate C-alleles. Thus, the induced “by-stander” aggregation pattern will not correspond to the design for the A-locus, but reflect aggregation patterns at the C-locus and will be predictable on the basis of the latter.

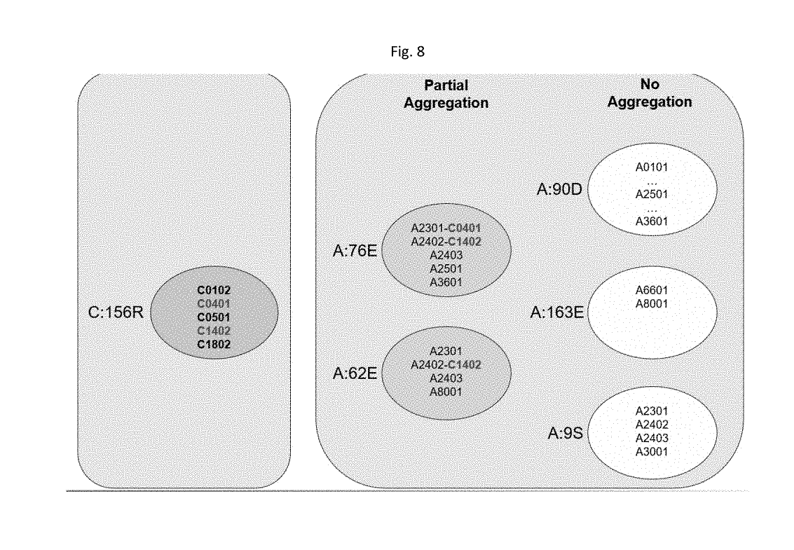

In particular, by-stander aggregation patterns can be detected by including, in the design at any specific locus, cells for any given allele at the specific locus of interest that have different alleles at other loci of interest. An example is shown in FIG. 8—anti C:156R will induce complete aggregation of the pool representing the “cognate” partition (along with partial aggregation in other C pools). In addition, however, anti-C:156R also will induce “by-stander” partial aggregation at certain A-locus (as well as B-locus) pools if these pools comprise C-alleles encoding the cognate antigenic determinant.

However, the aggregation pattern will not correlate with any A-locus partition and therefore can be recognized as a “by-stander” pattern. This is illustrated in FIG. 8: the pool A:76E which happens to comprise the cognate allele C0401 and pool A:62E which happens to comprise the cognate allele C1402 will undergo partial, but NOT complete, aggregation; while antibodies against any determinants encoded by any of A2301, A2402, A2403 may be ruled out as there will be no aggregation in pool A:9S which also comprises these A allele, and similarly antibodies against antigenic determinants encoded by A2501 or A3601 and by A8001 may be ruled by reference to pools A90D and A163E. Thus, the observed partial aggregation pattern may be recognized as a “by-stander” pattern.

Thus, in a preferred embodiment of the invention, cells for locus-specific pools will be selected so as to limit the range of alleles of these cells at other loci.

Further, the intended recipient's HLA type may provide additional indication of “by-stander” aggregation (see also Recipient-Specific Pools). For example, if the intended recipient has the allele A2402 which encodes A:62E as well as A:156E, then any aggregation observed in these partitions cannot be due to allo-anti-A:62E or allo-anti-A:156R.

By-stander aggregation may be eliminated by using suitably prepared microparticles or phospholipid vesicles displaying only a single type of affinity-purified or HLA molecule for constructing pools in accordance with pooling designs disclosed herein.

Serum Fractionation—

In serum containing both (i) an antibody directed to antigenic determinants encoded at the HLA-A locus (in class 1), and (ii) an antibody directed to a determinant encoded at the HLA-DR locus (class 2); if the pool were made from, say, leukocytes (expressing both class 1 and class 2), then the analysis would be simplified if the anti-class 1 antibody were removed first, by exposure to, say, platelets that do not express class 2 HLA. To fractionate the serum, one would expose it first to a collection of platelets (not necessarily in pools), to give the anti-class 1 antibody a chance to adsorb to the platelets, then analyze the “supernatant”—this would be an instance of affinity purification of the serum.

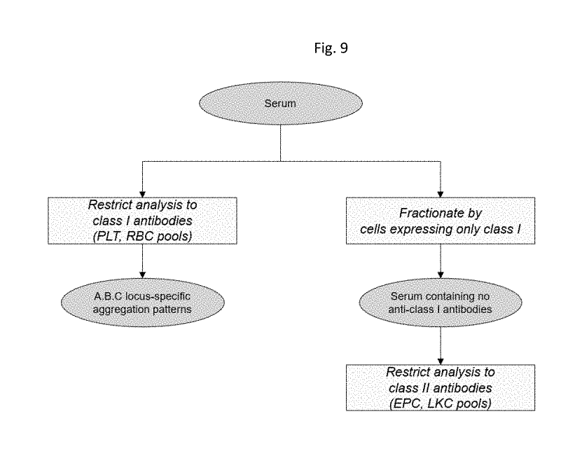

FIG. 9 illustrates restricting analysis to anti-class I, by using platelets and red cells that express class I but not class II (though these cells do express platelet- or red-cell specific antigens that also may induce allo-immunization) as well as cytotoxic T cells or endothelial cells (Canet2012). Conversely, to restrict analysis to anti-class II, such cells may be used as an affinity purification medium in order to adsorb anti-class I antibodies, leaving only (or primarily) anti-class II. A similar process could be used in fractionating serum suspected to contain both anti-RHCE and anti-RHD antibodies. For example, cells from an RHD negative individuals could be used for the fractionation of a serum suspected to contain both anti-RHCE and anti-RHD antibodies.

### EXAMPLES

### Example: “Private” Antigenic Determinants (See Also: FIG. 3)

Anticipating the encounter with patient sera comprising an antibody directed to the private antigenic determinant labeled by “6K” (or equivalently “243M”): form a pool preferably comprising cells that are homozygous for C0102 or less preferably, at least 2 (types of) cells with at least one C0102 allele but differing in their respective second allele. Exposure to an antibody directed against this antigenic determinant will induce aggregation of all such cells, aggregation of cells with one C0102 allele present in other pools and will leave suspended the cells in the complementary partition NOT containing C0102.

Private antigenic determinants may be recognized by the presence of a “1” in the partition count below each column; in special cases, a private antigenic determinant induces a bi-partition, as in the examples for the {1,15} cases shown to the very right of the matrix in FIG. 3.

The conceptually simplest way to construct a single-allele pool representing a private antigenic determinant will be to use homozygous cell lines (such as the IHWG Consanguineous Reference Panel). In practice, the combination of 2 heterozygous cell types which share the desired allele will be preferable, e.g. C0102-C0202 and C0102-C1701 would undergo complete aggregation when exposed to anti-9F.

Reaction patterns produced by commercially available “single antigen” bead sets (OneLambda-ThermoFisher) upon exposure to patient sera frequently produce reaction patterns wherein only one of the beads in the set is positive, and all others are negative. For example, with reference to FIG. 3, a reaction pattern with only C0102 positive, and all others negative, would indicate the presence of an antibody directed to an antigenic determinant labeled by the 1-aaSeqTag “6K” (or equivalently by “243M”). These simple reaction patterns, and only these, permit a direct inference as to the identity of the antibody giving rise to the positive response, all others require careful “decomposition” in terms of the true antigenic determinants: the allele partition construct introduced here provides the framework for such a decomposition.

### Example: Expected Pool Aggregation Pattern Produced by Antibodies to 77S and to 77N

FIG. 6 illustrates the expected aggregation pattern over a small set of pools comprising all pools generated by the 1-aa sequence tags at variable positions 116 (4 partitions), 156(4), 77(2), 9(4), 97(3) and 99(5). For anti-77S, the partition labeled by 77S will be expected to undergo complete aggregation, while its complement, labeled by 77N, will be expected to remain unaffected, along with several other partitions (shown without shading in FIG. 6) including 116L, 97M and 99G. All other partitions will be expected to undergo partial aggregation.

For anti-77N, the pattern will be the complement of that observed for 77S: the partition labeled by 77N will be expected to undergo complete aggregation, along with partitions 116L, 97M and 99G, while its complement, labeled by 77S, will be expected to remain unaffected, along with several other partitions (shown without shading in FIG. 5) including 116Y, 156Q, 9F, 99C and 99S; while all other partitions will be expected to undergo partial aggregation.

In the case of partial aggregation, the degree of aggregation (and hence the residual turbidity or equivalent measure) in each pool generally will reflect the number of “cognate” alleles (that is: allele expressing the “cognate” antigenic determinant) in the corresponding partition. These are high-lighted in boldface font: thus, for 77S, the partition “116F”, comprising only one allele expressing the cognate antigenic determinant, will be expected to display a lower degree of aggregation than, say, “116S”, comprising 5 (of a total of 7) such alleles, or 97W comprising 4 (of 5) such alleles. Thus, the pattern of residual turbidities (or equivalent measure of the degree of aggregation) may be matched to the predicted aggregation pattern.

The simple pool design shown in FIG. 6 has redundancies, i.e., 97M, 99G and 9F, 99C which may be eliminated as desirable.

Missing “Cognate” Partition—

It is instructive to consider the situation of encountering, say, anti-77S with a pool set that excludes cognate partitions for 77S and 77N. For anti-77S, pools 156Q (“C1601”), 9F (“C0102”), 99C (“C0102”) and 99S (“C0702”) still would be expected to undergo complete aggregation, as they each contain an allele that encodes the cognate antigenic determinant, and this pattern of complete aggregation, comprising three distinct alleles, indicates that the cognate partition may comprise additional alleles, and therefore that this partition may not have been represented in the pool set. Nevertheless, the observed aggregation pattern, even in the absence of 77S and 77N permits the inference that the cognate antigenic determinant is:
shared by alleles C0102, C0702 and C1601, and
not encoded by alleles C1502, C1802 (no aggregation).

Further, given partial aggregation in pools excluding C0102, C0702 and C1601, the cognate antigenic determinant also must be shared by at least one of:
C0401, C1402 (via 9S and 99F);
C0303, C0304 (via pool 116Y); and
C0401, C0501, C0801, C1701, C1802 (via 116F).

Thus, the list of candidate partitions (and hence the list of candidate antigenic determinants) can be pruned by inclusion and exclusion with reference to the allele partition dictionary, and one or more candidate partitions can be added to a future version of the pool set.

Selecting Cells from Non-aggregated Pools—

An important point to note is that even in the absence of complete aggregation in any pool, the partial aggregation pattern is clinically informative because the set of antigenic determinants expressed by cells in non-aggregated pools will not present a target to existing allo-antibodies and the constituent cells constitute candidates for administration to the intended recipient. In this way, the method of the present invention may be viewed as a specific parallel cross-match format comprising a multiplicity of candidate donor cells in accordance with the specific pooling design of the invention.

### Example: Expected Pool Aggregation Pattern Produced by Antibodies to 97M and to 97W

As illustrated in FIG. 7, anti-97M, directed against an antigenic determinant encoded by only C1802, will be expected to induce complete aggregate in pool 97M (and in the equivalent pool 99G) and no aggregation in the complementary partitions 97W and 97R. As the “cognate” antigenic determinant is a “private” antigenic determinant, many additional partitions will be expected to remain unaffected: only 116F, 156R, 77N and 9D will be expected to undergo partial aggregation which, given the presence of only one allele in each of these pools encoding the cognate antigenic determinant, may be minimal. In cases such as this, it may be advisable to add a secondary antibody (as in the standard indirect antiglobulin test) to amplify the aggregation. However, the large number of unaffected pools are as informative as the completely aggregated pools, as their union excluded only the allele 1802.

Anti-97W will be expected to generate a pool aggregation pattern exhibiting similar qualitative characteristics as those for anti-77S and anti-77N, namely: a fully aggregated “cognate” partition, along with several completely aggregated single-allele partitions; several partitions expected to undergo no aggregation (whose union in fact yields the complement of the cognate partition 97W) and thus will provide confirmation. In fact, in the absence of the cognate partition and its complement(s), the union of these non-aggregated pools would yield the complement of the cognate partition plus C0202, and thus would be almost determinative.

### Example: Estimated Inventory Size for Manufacturing Pool Sets

For any pool in a given design, each constituent cell must have at least one allele in the partition defining that pool, and to ensure the availability of such cells, the requisite inventory size should be such that, with a given probability, p, at least one cell type in the inventory has the least common allele in the partition; the partitions defined over a selected allele set. Denoting the frequency of the least common allele by f, the expected number of units of cells in inventory required to ensure at least one unit of cells having the corresponding haplotype is n=0.5*log(1−p)/log(1−f) where the factor of 0.5 reflects the fact that each cell carries two HLA alleles (at each locus).

Taking European A-B-C haplotype frequencies (in accordance with the National Marrow Donor Program compilation), with alleles abbreviated to 4 digits and aggregated accordingly, an expected inventory size of 68 cells ensures coverage for partition C:77N (least common allele: C1702, with frequency 0.0168) with probability 0.9, as illustrated in the table below along with other examples.

| | Least Common | Population | | Expected |
|---|---|---|---|---|
| Partition | Allele | Freq | Probability | Nof Cells |
| C: 77S | C0302 | 0.0065 | 0.9 | 172 |
| A: 90A | A7401 | 0.000725 | 0.7 | 831 |
| A: 90D | A4301 | 0.000145 | 0.7 | 4151 |
| A: 90D\A4301 | A3401 | 0.000724 | 0.7 | 831 |

In some cases, it is a single allele which requires a large inventory of cells, as in the case of allele A4301 in partition A:90D. In view of the general discussion of the probability of allo-exposure, such alleles may be dropped from the allele set without significant impact on the ability of the pool design to identify most of the expected allo-antibodies. Unless specific private antigenic determinants encoded by rare alleles are of special interest, pool designs may be limited to alleles exceeding a preset minimal population abundance; for example, a private antigenic determinant encoded by an allele with population frequency 0.005 would have an allo-exposure probability of only 0.0016 (or 0.16%) even at n=16. With such a limitation in place, pool designs will require not more than an inventory of 200-300 units randomly selected from the population, and possibly fewer by cultivating select donors with desirable HLA profiles. Locus-specific designs would draw from the same inventory in accordance with locus-specific designs.

### Example: Identifying Antigenic Determinants of RHCE and RHD

RHCE and RHD are trans-membrane proteins encoded by polymorphic genes (with a large and growing number of alleles) that are expressed on red blood cells. They display a multiplicity of antigenic determinants of which some are well known to be highly immunogenic. To identify antigenic determinants using the methods of the invention, proceed in analogy to the foregoing examples for HLA using the amino acid sequences of RHCE and RHD to construct allele partitions and using allele partitions to construct pools of cells. Compilations of alleles for RHCE and RHD are available at the website of the International Society for Blood Transfusion (“ISBT”).

### REFERENCES

All patents and patent applications in the foregoing text, and all references below, are hereby incorporated by reference.

### Non-Patent References

Buelow1995—Detection of panel-reactive anti-HLA class I antibodies by enzyme-linked immunosorbent assay or lymphocytotoxicity. Results of a blinded, controlled multicenter study. Buelow et al. Hum. Immunol. 1995; 44(1):1-11
El-Awar2009—Epitopes of HLA-A, B, C, DR, DQ, DP and MICA antigens. El Awar et al., Clin Transpl. 2009:295-321.
Canet2012—Profiling Posttransplant Circulating Antibodies in Kidney Transplantation Using Donor Endothelial Cells. Canet et al., Transplantation 2012—Volume 93—Issue 3 pp, 257-264
Everett1986—Class I HLA molecules on human erythrocytes. Quantitation and transfusion effects. Everett et al., Transplantation. 1987; 44(1):123-9.
Einecke2009—Antibody-mediated microcirculation injury is the major cause of late kidney transplant failure. Elnecke et al., Am J Transplant. 2009; 9(11):2520-31
Engelhard1978—Reconstitution of purified detergent-soluble HLA-A and HLA-B antigens into phospholipid vesicles. Engelhard et al., Proc. Nati. Acad. Sci. USA Vol. 75, No. 11, pp. 5688-5691, 1978
Fidler2012—Crossmatching by complement-dependent lymphocytotoxicity. Fidler et al., Methods Mol Biol. 2012; 882:359-77
Leffell2014—Red blood cell transfusions and the risk of allosensitization in patients awaiting primary kidney transplantation. Leffell et al., Transplantation. 2014; 97(5):525-33
Maiers2002—A computations approach to serologic epitope mapping; Chapt G1.2 (p. 886) in: 13th IHWS Serology and HLA Phenotypes Joint Report
McPherson2010—Red Blood Cell Transfusions Are Associated with HLA Class I but not H-Y Alloantibodies in Children with Sickle Cell Disease. McPherson et al., Br J Haematol. 2015; 170(2): 247-256
Nambiar2006—HLAMatchmaker-driven analysis of responses to HLA-typed platelet transfusions in alloimmunized thrombocytopenic patients. Nambiar et al., Blood. 2006; 107(4): 1680-1687
Nickel2015—Immunophenotypic parameters and RBC alloimmunization in children with sickle cell disease on chronic transfusion. Nickel et al., Am J Hematol. 2015; 90(12):1135-41
Pei2003—Single human leukocyte antigen flow cytometry beads for accurate identification of human leukocyte antigen antibody specificities. Pei et al., Transplantation. 2003 15; 75(1):43-9
Reed2013—Comprehensive Assessment and Standardization of Solid Phase Multiplex-Bead Arrays for the Detection of Antibodies to HLA Reed et al., Am J Transplant. 2013; 13(7): 1859-1870.
Sellares2012—Understanding the causes of kidney transplant failure: the dominant role of antibody-mediated rejection and nonadherence. Sellares et al., Am J Transplant. 2012; 12(2):388-99
Sintnicolaas1995—Leukocyte Depletion of Random Single-Donor Platelet Transfusions Does Not PreventSecondary Human Leukocyte Antigen-Allolmmunization and Refractoriness: A Randomized Prospective Study. Sintnicolaas et al., Blood, Vol 85, No 3, 1995: pp 824-828
TRAP1997—Leukocyte Reduction and Ultraviolet B Irradiation of Platelets to Prevent Alloimmunization and Refractoriness to Platelet Transfusions. Trap et al., N Engl J Med 1997; 337:1861-1870
Vermehren2002—Isolation of precursor endothelial cells from peripheral blood for donor-specific cross-matching before organ transplantation Vermehren et al., Transplantation. 2002; 74(11):1479-86
Zachary2009—Using real data for a virtual crossmatch. Zachary et al., Hum Immunol. 2009; 70(8):574-9

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The defined terms in the Definitions section above are to be applied in interpreting such terms in claims. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A device for identifying suitable donors of blood, blood products, cells or organs, comprising: a multiplicity of pools of cells, wherein different cells for each pool are selected to represent a designated group of alleles at a genetic locus encoding an antigen of interest, such that all alleles in the group encode the same amino acids at one or more designated variable positions of the amino acid sequence of the antigen.

2. The device of claim 1 further including in the series of wells, serum from a potential recipient, or samples derived from said serum.

3. The device of claim 1 wherein the pools of cells are HLA class I or HLA class II expressing cells.

4. The device of claim 1 wherein the pools of cells are RHCE or RHD expressing cells.

5. The device of claim 1 further including a labeled secondary antibody directed to the Fc portion of an IgG class antibody.

6. The device of claim 5 where the number of designated variable positions is three or less, where the combination of variable positions for different antigens of interest encodes the antigens of interest.

7. A method identifying cognate antigenic determinants of antibodies in serum from an individual, to be used in identifying donors of blood, blood products, cells or organ samples for said individual, comprising:
(i) preparing a multiplicity of pools of cells, wherein the cells for each pool are selected to represent a designated group of alleles at a genetic locus encoding an antigen of interest, such that the alleles in the group all encode the same amino acids at one or more designated variable positions of the amino acid sequence of the antigen;
(ii) dispensing aliquots of serum from said individual into the pools;
(iii) monitoring the degree of aggregation of cells in each pool, where a greater or lesser degree of aggregation in a pool indicates that a greater or lesser proportion of the cells in that pool share the cognate antigenic determinants of the antigens recognized by antibodies in the serum;
(iv) ranking pools in order of the degree of aggregation;
(v) recording the rankings as an indicator of which pools contain, and therefore which alleles encode, cognate antigenic determinants to antibodies in said serum; and
(vi) selecting suitable donors for the individual from potential donors whose alleles are in the group of alleles for pools with the lesser rankings by degree of aggregation.

8. The method of claim 7 wherein the cognate antigenic determinants of the pool displaying a greater degree of aggregation, in turn identify the specificity of an antibody in the serum.

9. The method of claim 8 wherein a degree of aggregation above a preset threshold in any pool indicates the presence of the antibody in the serum.

10. The method of claim 7 wherein a population of non-aggregated cells in the highest-ranked pool indicates that none of the pools contain cognate antigenic determinants for any antibodies in the serum.

11. The method of claim 7 wherein a population of aggregated cells in the lowest-ranked pool provides an indication that none of the pools represent a group of alleles not encoding a cognate antigenic determinant recognized by an antibody in the serum.

12. The method of claim 7 wherein if the highest-ranked pool contains a population of non-aggregated cells, or the lowest-ranked pool contains a population of aggregated cells, new pools containing cells having additional alleles encoding additional antigenic determinants are prepared and steps (ii) to (vi) above are repeated using said new pools.

13. The method of claim 7 wherein each pool is triplicated for the purpose of averaging over the aggregated fraction of cells in pools with cells from the same individual(s).

14. The method of claim 7 wherein the cells in the pools are HLA class I or HLA class II expressing cells.

15. The method of claim 7 wherein the cells in the pools are RHCE or RHD expressing cells.

16. The method of claim 7 wherein the serum is first fractionated by exposure to anti-gene or anti-locus specific antibodies.

17. The method of claim 7 wherein aggregation is facilitated by addition to the pools of a secondary antibody directed to the Fc portion of the antibodies in the serum.

18. The method of claim 7 wherein the secondary antibody is fluorescently labeled and pools are ranked by mean intensity of fluorescence.

19. The method of claim 7 wherein the ranking of pools is compared to a predicted ranking in order to determine the identity of the cognate antigenic determinant for an antibody in the serum.

20. The method of claim 18 wherein the ranking of pools by fluorescence is compared to a predicted ranking in order to determine the identity of the cognate antigenic determinant for an antibody in the serum.

21. The method of claim 7 wherein the same amino acids at one or more designated variable positions of the amino acid sequence of the antigen are encoded by a tag representing said same amino acids.

22. The method of claim 7 wherein the allele partitions represented by pools exclude allele partitions of the intended recipient.

23. The method of claim 7 wherein cells different prospective donors are sequentially added to pools and the aggregation response is assessed following each addition.

24. The method of claim 7 further including the step of administering cells to the recipient from a potential donor whose cells were not aggregated by the recipient serum.
